SEQUENCE LISTING (31 sequences)

SEQ ID NO: 1 - DNA, 45 bp, Artificial Sequence
  synthetic oligonucleotide for assembly of 594-base nucleic acid molecule of SEQ ID NO31
atgaccttcttcgaatctctcgcgcgcaaactggaccgcaccggt

SEQ ID NO: 2 - DNA, 44 bp, Artificial Sequence
  synthetic oligonucleotide for assembly of 594-base nucleic acid molecule of SEQ ID NO31
gcgtgccaaacggtcgggtcaacaccggtgcggtccagtttgcg

SEQ ID NO: 3 - DNA, 45 bp, Artificial Sequence
  synthetic oligonucleotide for assembly of 594-base nucleic acid molecule of SEQ ID NO31
gttgacccgaccgtttggcacgcgccgcctccgccacctccgacc

SEQ ID NO: 4 - DNA, 45 bp, Artificial Sequence
  synthetic oligonucleotide for assembly of 594-base nucleic acid molecule of SEQ ID NO31
ataccgttcggagccagaacaccggtcggaggtggcggaggcggc

SEQ ID NO: 5 - DNA, 45 bp, Artificial Sequence
  synthetic oligonucleotide for assembly of 594-base nucleic acid molecule of SEQ ID NO31
ggtgttctggctccgaacggtatgctggacgacgcgcaccgttgg

SEQ ID NO: 6 - DNA, 45 bp, Artificial Sequence
  synthetic oligonucleotide for assembly of 594-base nucleic acid molecule of SEQ ID NO31
ggaccttcaccagccggcagggtccaacggtgcgcgtcgtccagc

SEQ ID NO: 7 - DNA, 45 bp, Artificial Sequence
  synthetic oligonucleotide for assembly of 594-base nucleic acid molecule of SEQ ID NO31
accctgccggctggtgaaggtccggaagacgttgcggttgaccac

SEQ ID NO: 8 - DNA, 43 bp, Artificial Sequence
  synthetic oligonucleotide for assembly of 594-base nucleic acid molecule of SEQ ID NO31
accggtaacaacacgaccgtcgtggtcaaccgcaacgtcttcc

SEQ ID NO: 9 - DNA, 43 bp, Artificial Sequence
  synthetic oligonucleotide for assembly of 594-base nucleic acid molecule of SEQ ID NO31
gacggtcgtgttgttaccggtggcaacgatggccgtatttggc

SEQ ID NO: 10 - DNA, 42 bp, Artificial Sequence
  synthetic oligonucleotide for assembly of 594-base nucleic acid molecule of SEQ ID NO31
gtgaccgtgaccgtcgaaacgccaaatacggccatcgttgcc

SEQ ID NO: 11 - DNA, 44 bp, Artificial Sequence
  synthetic oligonucleotide for assembly of 594-base nucleic acid molecule of SEQ ID NO31
gtttcgacggtcacggtcacgcgacggaactcgctaatacccac

SEQ ID NO: 12 - DNA, 45 bp, Artificial Sequence
  synthetic oligonucleotide for assembly of 594-base nucleic acid molecule of SEQ ID NO31
ttctacacccaaagggcggccgtgggtattagcgagttccgtcgc

SEQ ID NO: 13 - DNA, 45 bp, Artificial Sequence
  synthetic oligonucleotide for assembly of 594-base nucleic acid molecule of SEQ ID NO31
ggccgccctttgggtgtagaaatcctggacgacggtcgtttcctg

SEQ ID NO: 14 - DNA, 45 bp, Artificial Sequence
  synthetic oligonucleotide for assembly of 594-base nucleic acid molecule of SEQ ID NO31
accacgttccgcgtcgcagatcaggaaacgaccgtcgtccaggat

SEQ ID NO: 15 - DNA, 45 bp, Artificial Sequence
  synthetic oligonucleotide for assembly of 594-base nucleic acid molecule of SEQ ID NO31
atctgcgacgcggaacgtggtgttctccgtgttgacgaccagggt

SEQ ID NO: 16 - DNA, 45 bp, Artificial Sequence
  synthetic oligonucleotide for assembly of 594-base nucleic acid molecule of SEQ ID NO31
atcagcgagaacgtcaacgcgaccctggtcgtcaacacggagaac

SEQ ID NO: 17 - DNA, 44 bp, Artificial Sequence
  synthetic oligonucleotide for assembly of 594-base nucleic acid molecule of SEQ ID NO31
cgcgttgacgttctcgctgatgcggctgcaggtcgtccactcgt

SEQ ID NO: 18 - DNA, 42 bp, Artificial Sequence
  synthetic oligonucleotide for assembly of 594-base nucleic acid molecule of SEQ ID NO31
cgcagagttgttgcacgcaacgagtggacgacctgcagccgc

SEQ ID NO: 19 - DNA, 45 bp, Artificial Sequence
  synthetic oligonucleotide for assembly of 594-base nucleic acid molecule of SEQ ID NO31
tgcgtgcaacaactctgcggtcggccgtgacggtatcgtttactt

SEQ ID NO: 20 - DNA, 45 bp, Artificial Sequence
  synthetic oligonucleotide for assembly of 594-base nucleic acid molecule of SEQ ID NO31
gtgcgcagaagagtcggtgaagtaaacgataccgtcacggccgac

SEQ ID NO: 21 - DNA, 43 bp, Artificial Sequence
  synthetic oligonucleotide for assembly of 594-base nucleic acid molecule of SEQ ID NO31
caccgactcttctgcgcacttcaccatcgcggaccaccgttac

SEQ ID NO: 22 - DNA, 45 bp, Artificial Sequence
  synthetic oligonucleotide for assembly of 594-base nucleic acid molecule of SEQ ID NO31
accacggtgttccaacaggtcgtaacggtggtccgcgatggtgaa

SEQ ID NO: 23 - DNA, 45 bp, Artificial Sequence
  synthetic oligonucleotide for assembly of 594-base nucleic acid molecule of SEQ ID NO31
gacctgttggaacaccgtggtaccggtcgtctgctgcgtctggac

SEQ ID NO: 24 - DNA, 44 bp, Artificial Sequence
  synthetic oligonucleotide for assembly of 594-base nucleic acid molecule of SEQ ID NO31
tcagtttcaccggtacgcgggtccagacgcagcagacgaccggt

SEQ ID NO: 25 - DNA, 40 bp, Artificial Sequence
  synthetic oligonucleotide for assembly of 594-base nucleic acid molecule of SEQ ID NO31
ccgcgtaccggtgaaactgacctgctcgctgagggtctgc

SEQ ID NO: 26 - DNA, 40 bp, Artificial Sequence
  synthetic oligonucleotide for assembly of 594-base nucleic acid molecule of SEQ ID NO31
accaacaccgttcgcgaactgcagaccctcagcgagcagg

SEQ ID NO: 27 - DNA, 60 bp, Artificial Sequence
  synthetic oligonucleotide for assembly of 594-base nucleic acid molecule of SEQ ID NO31
gagggtctgcagttcgcgaacggtgttggtatgaccttcttcgaatctctcgcgcgcaaa

SEQ ID NO: 28 - DNA, 28 bp, Artificial Sequence
  synthetic oligonucleotide for assembly of 594-base nucleic acid molecule of SEQ ID NO31
agcgagcaggtcagtttcaccggtacgc

SEQ ID NO: 29 - DNA, 32 bp, Artificial Sequence
  synthetic oligonucleotide for assembly of 594-base nucleic acid molecule of SEQ ID NO31
ctggaccgcaccggtgttgacccgaccgtttg

SEQ ID NO: 30 - DNA, 60 bp, Artificial Sequence
  synthetic oligonucleotide for assembly of 594-base nucleic acid molecule of SEQ ID NO31
tttgcgcgcgagagattcgaagaaggtcataccaacaccgttcgcgaactgcagaccctc

SEQ ID NO: 31 - DNA, 594 bp, Artificial Sequence
  synthetic oligonucleotide for demonstration purposes only, not based on any naturally occurring sequence
atgaccttcttcgaatctctcgcgcgcaaactggaccgcaccggtgttgacccgaccgtttggcacgcgccgcctccgccacctccgaccggtgttctggctccgaacggtatgctggacgacgcgcaccgttggaccctgccggctggtgaaggtccggaagacgttgcggttgaccacgacggtcgtgttgttaccggtggcaacgatggccgtatttggcgtttcgacggtcacggtcacgcgacggaactcgctaatacccacggccgccctttgggtgtagaaatcctggacgacggtcgtttcctgatctgcgacgcggaacgtggtgttctccgtgttgacgaccagggtcgcgttgacgttctcgctgatgcggctgcaggtcgtccactcgttgcgtgcaacaactctgcggtcggccgtgacggtatcgtttacttcaccgactcttctgcgcacttcaccatcgcggaccaccgttacgacctgttggaacaccgtggtaccggtcgtctgctgcgtctggacccgcgtaccggtgaaactgacctgctcgctgagggtctgcagttcgcgaacggtgttggt

(19) United States
(12) Patent Application Publication
(10) Pub. No.: US 2009/0305359 A1
(43) Pub. Date: Dec. 10, 2009

(54) METHOD FOR PRODUCING CIRCULAR DUPLEX POLYNUCLEOTIDES FROM LINEAR DUPLEX POLYNUCLEOTIDES AND APPLICATIONS THEREOF

(76) Inventors: G. Wesley Hatfield, Corona del Mar, CA (US); Liza S.Z. Larsen, Irvine, CA (US); Richard H. Lathrop, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/466,236

(22) Filed: May 14, 2009

Related U.S. Application Data
(60) Provisional application No. 61/053,638, filed on May 15, 2008.

(51) Int. Cl. C12P 19/34; C12N 15/87; C07H 21/00

(52) U.S. Cl. 435/91.52; 435/91.2; 435/455; 536/25.3

(57) ABSTRACT

Methods are provided for making a circular duplex polynucleotide. Such methods can include providing a mixture of sequence specific linear duplex polynucleotides and denaturing and reannealing the polynucleotide mixture under conditions such that some of the polynucleotides form circular duplexes that comprise the desired polynucleotide.

### BACKGROUND

1. Technical Field

This invention relates to a novel method of preparing circular duplex polynucleotides, and methods of using such circular duplex polynucleotides. Methods of the present invention can be used to prepare a desired polynucleotide that has few, if any, nucleotide sequence errors. Methods of the present invention also can be used to clone polynucleotides in host cells. The invention also relates to a polynucleotide prepared using the methods, and to a peptide or a polypeptide encoded by the polynucleotide.

2. Description of the Related Art

Advances in biotechnology have led to the use of “designer” or synthetic genes for many medical, agricultural, and bioindustrial applications. While the use of synthetic genes is now commonplace, current methods for making these genes can limit their utility. For example, one current method for producing a synthetic gene involves making a set oligonucleotides and then ligating the oligonucleotides together in the proper order to create the full length synthetic gene. Depending on the underlying chemical synthesis error rate, approximately 1 in 5 or 1 in 10 oligonucleotides of about 45 nucleotides long will contain an error, typically in the form of a deletion. While some of these sequence errors may be “silent” and thus do not affect the amino acid sequence of the polypeptide produced from the gene, the majority of these sequence errors change the amino acid sequence of the polypeptide and may thus render the synthetic gene useless.

A number of methods have been employed to rapidly prepare synthetic genes and to decrease the rate of sequence errors occurring in oligonucleotide and polynucleotide synthesis.

Fuhrmann et al. (Nucleic Acids Res., 33(6):e58 [2005]) describe the use of certain endonucleases to cleave single base pair mismatches, insertions, and deletions in synthetic genes and oligonucleotides.

Bang and Church (Nat. Methods, 5(1):37-39 [2008]) describe a method of gene synthesis by circular assembly amplification. The authors report that this method improves gene synthesis quality. A limitation of this method is that single-stranded oligonucleotides are used as starting materials, which limitation is overcome by the present invention teaching how to use linear duplex polynucleotides as starting materials.

Geu-Flores et al. (Nucleic Acids Res., 35(7):e55 [2007]) describe cloning of multiple PCR (polymerase chain reaction) products using PCR primers that contain deoxyuridine bases near the 5′ ends. According to the authors, the method allows for simultaneous fusion and cloning of the PCR products. A limitation of this method is that special-purpose PCR primers that contain special-purpose bases must be used, which limitation is overcome by the present invention teaching how to use standard PCR primers with standard DNA bases for cloning.

While each of these methods has proven somewhat useful, the problem remains of rapidly generating synthetic genes that can easily be cloned in host cells and have few or no nucleotide sequence errors.

It is thus an object of the present invention to provide synthetic genes with significantly fewer nucleotide sequence errors as compared with many other known methods.

It is a further object of the present invention to provide a means for rapidly generating a synthetic gene.

It is still another object of the present invention to simplify the process of constructing a synthetic polynucleotide by preparing it as a circular duplex polynucleotide.

These and other objects will be apparent to the ordinary skilled artisan from the disclosure herein.

### SUMMARY

In one embodiment, the invention is directed to a method of making a circular duplex polynucleotide that comprises a desired polynucleotide, the method comprising providing a mixture of sequence specific linear duplex polynucleotides (defined below) and denaturing and reannealing the polynucleotide mixture under conditions such that some of the polynucleotides form circular duplexes that comprise the desired polynucleotide.

In another embodiment, the invention relates to a method of reducing nucleotide sequence errors in a pool of desired polynucleotides, comprising providing a mixture of sequence specific linear duplex polynucleotides, denaturing and reannealing the polynucleotide mixture under conditions such that some of the polynucleotides form circular duplexes that comprise the desired polynucleotide, ligating circular duplexes using a ligase such as T4 DNA ligase to form covalently linked circular duplex polynucleotides, and destroying linear polynucleotides and circular polynucleotides containing nucleotide sequence errors. Optionally, the desired polynucleotides can be amplified via PCR or polymerase extension. In certain embodiments, amplification is accomplished by linearizing the circular duplexes using a restriction enzyme, then using PCR or polymerase extension.

The invention further includes a method of cloning a desired polynucleotide, comprising providing a mixture of sequence specific linear duplex polynucleotides, denaturing and reannealing the polynucleotide mixture under conditions such that some of the polynucleotides form circular duplexes that comprise the desired polynucleotide, and then transforming the polynucleotide mixture into a host cell.

The invention further includes a method of reducing nucleotide sequence errors in a mutational library wherein a target desired polynucleotide is mutagenized across a discrete region, comprising providing a mixture of linear duplex polynucleotides that include two circular permutations of the desired polynucleotide minus said mutagenesis region, one with and one without flanking regions surrounding the mutagenesis region; providing a mixture of single-stranded mutagenesis polynucleotides, each containing a variable mutagenesis region surrounded by said flanking regions; mixing, denaturing, and reannealing said mixtures under conditions such that some of the polynucleotides form circular duplex polynucleotides except that the mutagenesis region is single-stranded; extending the complementary strand across the single-stranded mutagenesis region by using a non-displacing polymerase; ligating circular duplexes using a ligase such as T4 DNA ligase to form covalently linked circular duplex polynucleotides; and destroying linear polynucleotides and circular polynucleotides containing nucleotide sequence errors. Optionally, the target desired polynucleotide may be mutagenized across several distinct discrete regions by combining fragments each of which has been mutagenized across one discrete region as described.

In certain embodiments of the invention, the denaturing and reannealing conditions for the polynucleotides comprise heating and then cooling the polynucleotide mixture. Optionally, the heating is conducted at a temperature from about 80-120 degrees Celsius for about 5-15 minutes, followed by cooling to about 10-20 degrees at a rate of about 0.5-2 degrees Celsius per minute.

Also optionally, heating is at about 98 degrees Celsius for about 2 minutes, followed by cooling to about 16 degrees Celsius at a rate of about one degree per minute.

In other embodiments of the invention, the undesirable linear polynucleotides are removed from the circular duplex polynucleotides by destroying the linear polynucleotides using an exonuclease. Optionally, the exonuclease is selected from the group consisting of lambda exonuclease, T7 exonuclease, exonuclease I, exonuclease III, and RecJ exonuclease.

In another embodiment of the invention, the circular duplex polynucleotides having nucleotide mismatches are removed using an endonuclease. Optionally, the endonuclease is selected from the group consisting of endonuclease V, Bal 31 endonuclease, and mung bean nuclease.

In still other embodiments of the invention, the circular duplex polynucleotides containing nucleotide mismatches are removed using an endonuclease in the presence of an exonuclease. Optionally, the exonuclease is selected from the group consisting of lambda exonuclease, T7 exonuclease, exonuclease I, exonuclease III, and RecJ exonucleases, and the endonuclease is selected from the group consisting of endonuclease V, Bal 31 endonuclease, and mung bean nuclease.

In yet other embodiments, the invention is directed to a polynucleotide made by any of the methods of the invention.

In still another embodiment, the invention relates to a polypeptide encoded by any of the polynucleotides of the invention.

### BRIEF DESCRIPTION OF THE DRAWINGS

FIG. 1 is a schematic diagram of the design of the sequence specific linear duplex polynucleotides used to prepare a 594 base pair gene as described in Example 1.

FIG. 2 sets forth the sequences of the oligonucleotides used to prepare the circular polynucleotide molecules in the Examples.

FIG. 3 sets forth the nucleotide sequence of the 594 base pair desired polynucleotide.

FIG. 4 is a schematic diagram of the design of the sequence specific linear duplex polynucleotides used to prepare a circular duplex polynucleotide comprising a desired polynucleotide containing adjacent regions A, B, C, and D, which serve for, e.g., primer complementarity or other purposes.

FIG. 5 is a schematic diagram of the insertion of the sequence specific linear duplex polynucleotides from FIG. 4 into a plasmid.

FIG. 6 is a schematic diagram of the design and use of sequence specific linear duplex polynucleotides used to reduce nucleotide sequence errors in a mutational library wherein a target desired polynucleotide is mutagenized across a discrete region.

FIG. 7 is a schematic diagram of completion of a duplex circular polynucleotide of FIG. 6, using a non-displacing polymerase.

FIG. 8 sets forth a non-limiting example of a simple enzyme-free cloning method and cloning kit.

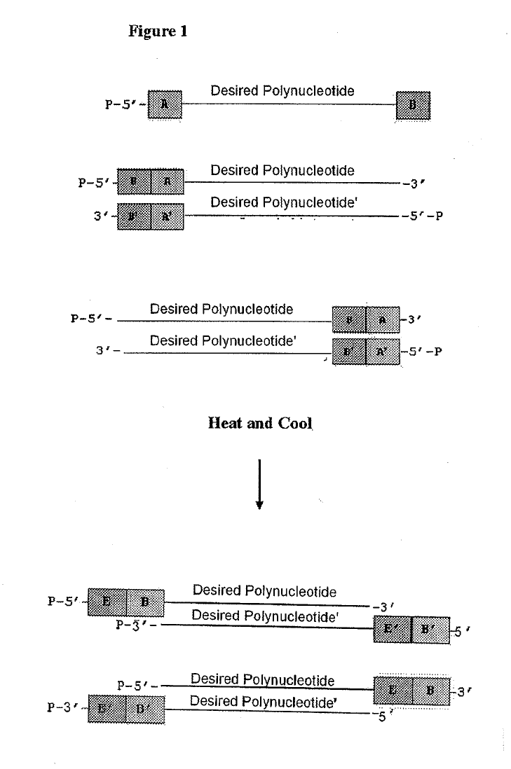

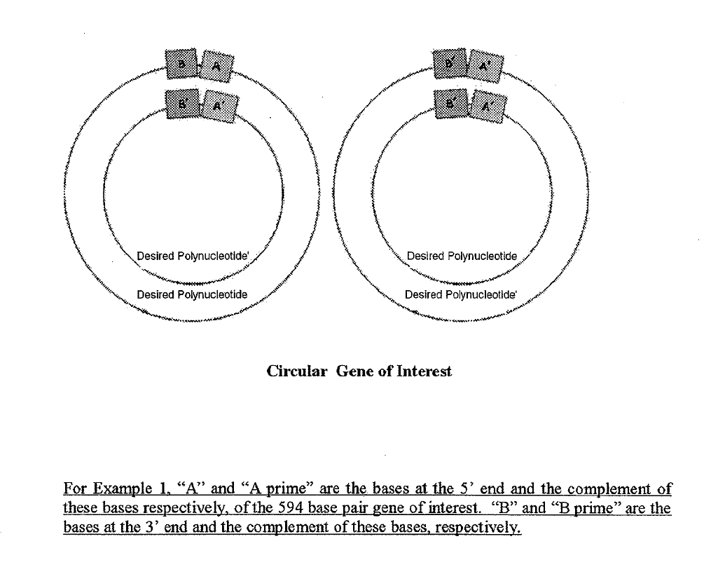

### DETAILED DESCRIPTION

The present invention provides a novel method of creating circular duplex polynucleotides from a mixture of linear duplex polynucleotides. The invention further provides a method for rapidly generating a synthetic duplex polynucleotide of interest (referred to herein as a “desired polynucleotide”) that has few, if any, nucleotide sequence errors. Some embodiments of the invention provide an enzyme-free method of cloning a desired polynucleotide into a host cell, and an enzyme-free cloning kit that is simpler and easier to use than current cloning kits.

A feature of the invention is the development of specific sequence and overlap constraints on the linear duplex polynucleotides used as starting materials (“sequence specific linear duplex polynucleotides” as discussed below). These constraints permit the starting materials to form circular duplex polynucleotides with no more complicated operation than heating, cooling, and ligating, or, optionally, heating, cooling, polymerizing and ligating.

The linear duplex polynucleotides used as starting materials may have either blunt or over-hanging (“sticky”) ends, and can be made using well known techniques routinely used to generate linear duplex polynucleotides, including but not limited to complementary DNA (“cDNA”) cloning, polymerase chain reaction (“PCR”), and restriction enzyme or mechanical stress cleavage of natural DNA. The invention eliminates the requirement for single-stranded oligonucleotides to serve as starting materials. The invention also eliminates the need for special-purpose enzymes, the complicated steps of special-purpose enzymatic reactions, and the dependence on special-purpose primers, which some current methods require.

The present invention enables polynucleotide synthesis with reduced sequence error rates as compared with conventional polynucleotide synthesis procedures. The circular duplex polynucleotides of the present invention are treated using enzymatic error reduction methods. Certain endonucleases are used to detect and nick base pairs that do not exhibit perfect duplex hybridization, and certain exonucleases are used to destroy nicked or linear polynucleotides in the mixture. In one embodiment of the invention, the circular duplex polynucleotides can be transformed directly into a host organism, for example, if the desired polynucleotide is a plasmid. In some such embodiments, the circular duplex polynucleotides are transformed without ligation of any nicks in the circular duplex. In other such embodiments, the circular duplex polynucleotides are first ligated to seal nicks, then treated with exonuclease(s) and endonuclease(s) to reduce errors, and finally transformed into a host organism. Alternatively, the resultant duplex polynucleotides can be amplified using PCR or any other suitable method to produce a desired polynucleotide. PCR products created by a high-fidelity polymerase are known to have generally a low copy error rate, and thus the desired polynucleotides generated by PCR have few, if any, nucleotide sequence errors. Thus, this invention eliminates the need for the successive rounds of nucleotide sequencing traditionally required during synthetic gene production to identify low-error or error-free gene fragments, and does so using a simpler and more efficient process than many methods well known in the art. As such, the present invention saves significant time, labor, and money.

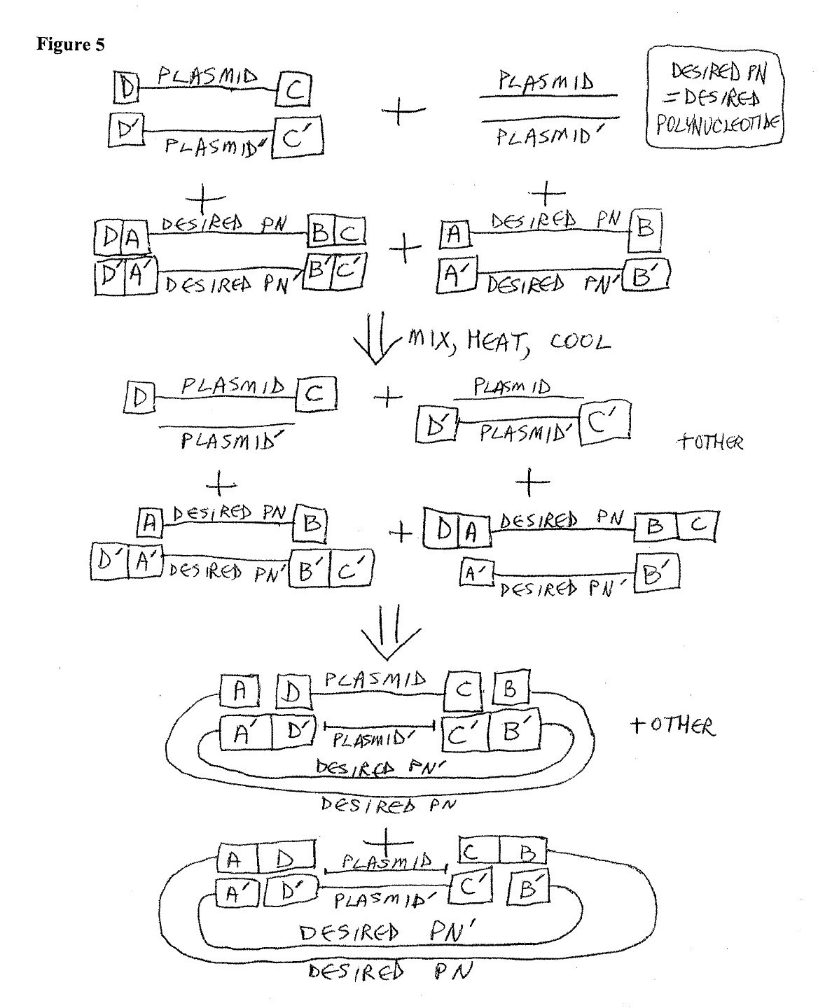

The present invention also provides a simple enzyme-free cloning method and cloning kit. The cloning uses specific primers and plasmid fragments, and is exemplified in FIG. 5. Two PCR reactions on any desired duplex polynucleotide, using specific combinations of these primers, typically yield PCR products that, when combined with the plasmid fragments, constitute sequence-specific linear duplex polynucleotides (as defined below). After heating and cooling, the resulting circularized duplex comprises both the desired polynucleotide and the plasmid into which it was inserted, and can be transformed directly into host cells. The necessary reagents can be supplied in kit form, in one embodiment as three tubes containing linear duplex DNA. That embodiment of the method for cloning with the kit includes: (1) two PCR reactions, (a) one with the desired polynucleotide and tube#1, and (b) the other with the desired polynucleotide and tube#1 and tube#2; (2) combining the PCR products; (3) performing a PCR clean-up; (4) adding tube#3; (5) heating and cooling; (6) transforming the mixture into cells. Cloning efficiency is high. The method and kit are simpler, faster, and cheaper than current methods. As such, the present invention saves significant time, labor, and money.

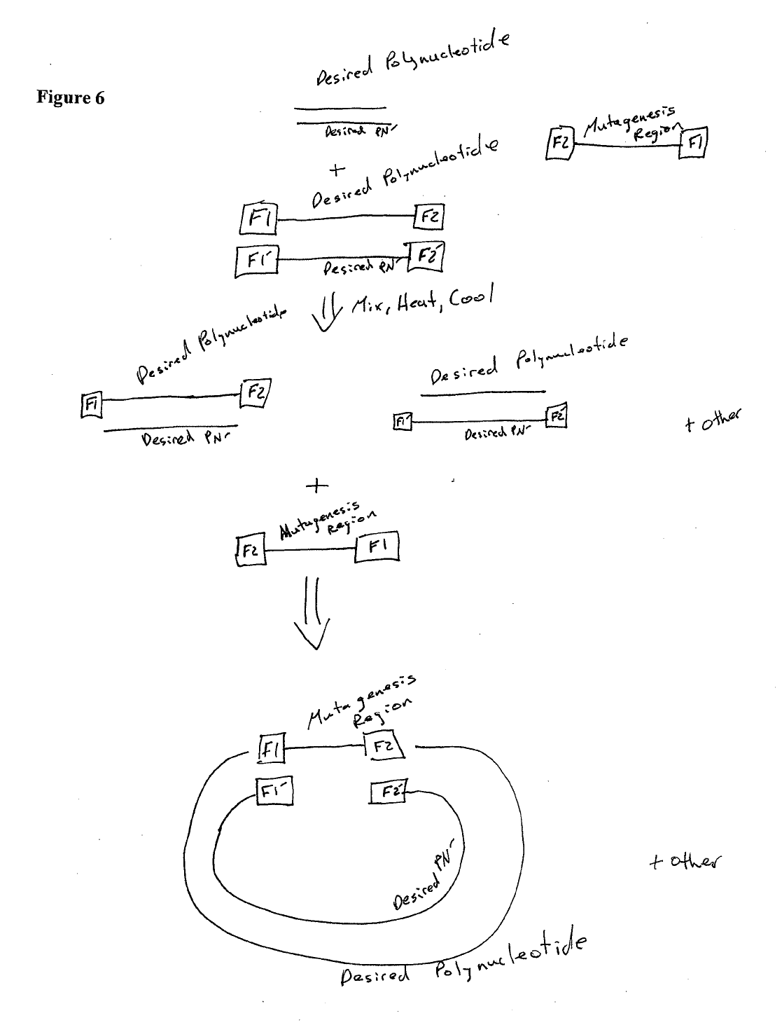

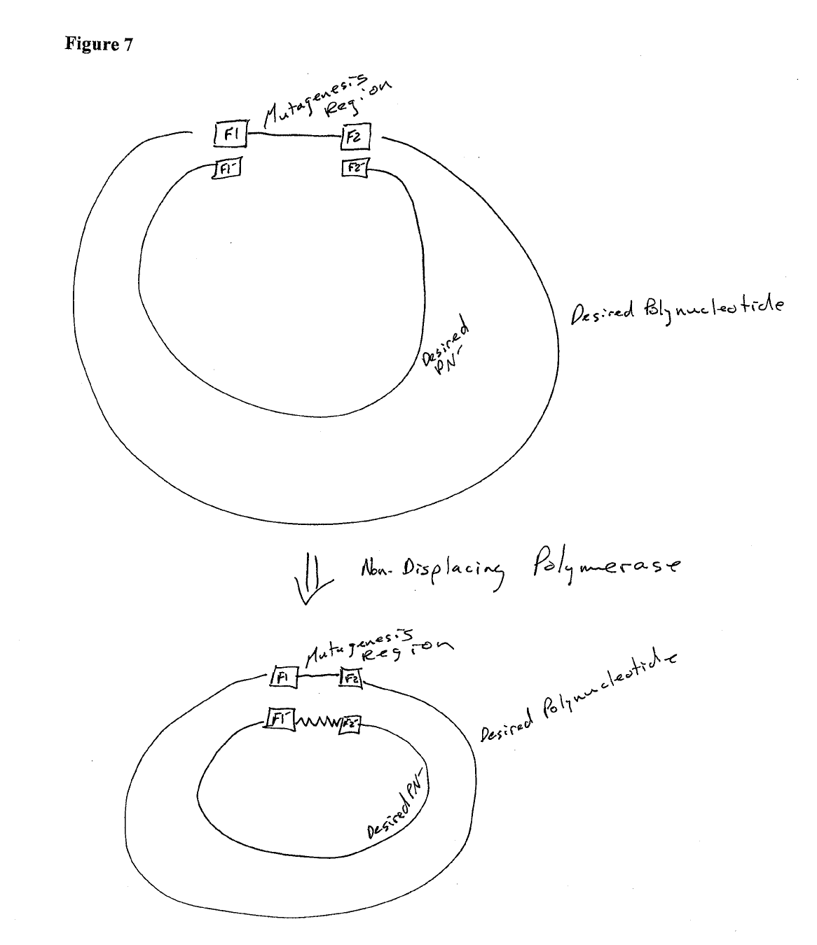

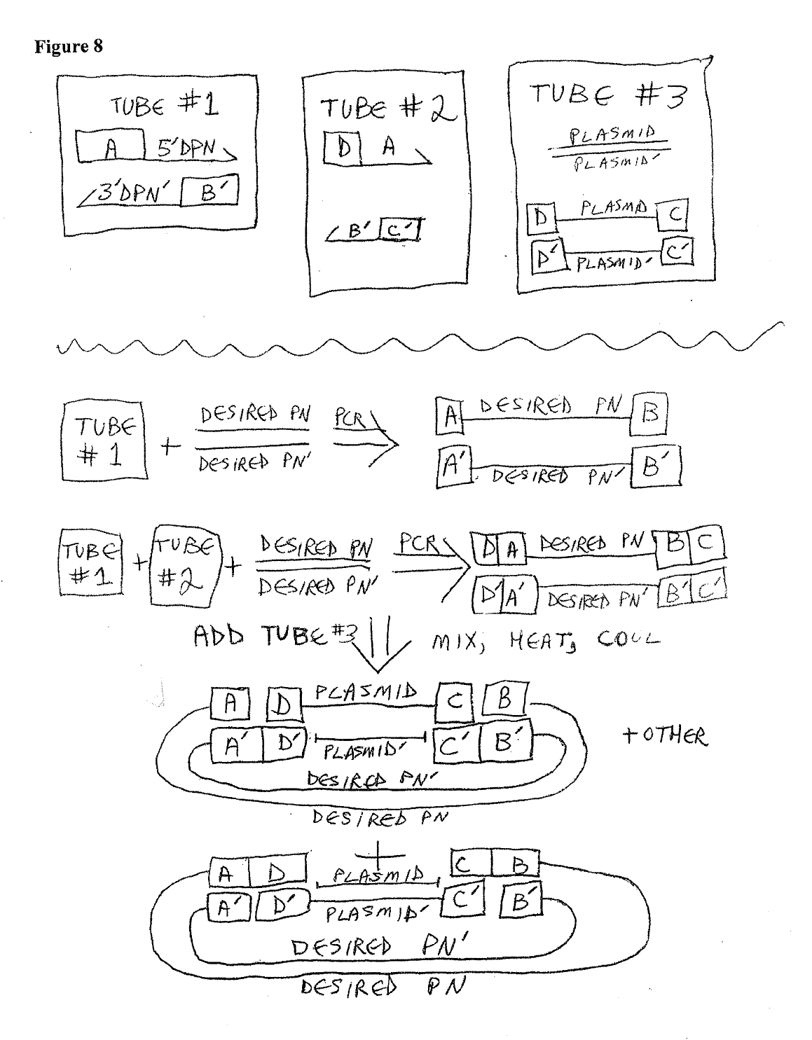

The invention further includes a method of reducing nucleotide sequence errors in a mutational library wherein a target desired polynucleotide is mutagenized across a discrete region (see, e.g., FIGS. 6 and 7), comprising providing a mixture of linear duplex polynucleotides that include two circular permutations of the desired polynucleotide minus said mutagenesis region, one with and one without flanking regions (e.g., F1 and F2) surrounding the mutagenesis region; providing a mixture of single-stranded mutagenesis polynucleotides, each containing a variable mutagenesis region surrounded by said flanking regions; mixing, denaturing, and reannealing said mixtures under conditions such that some of the polynucleotides form circular duplex polynucleotides except that the mutagenesis region is single-stranded; extending the complementary strand across the single-stranded mutagenesis region by using a non-displacing polymerase; ligating circular duplexes using a ligase such as T4 DNA ligase to form covalently linked circular duplex polynucleotides; and destroying linear polynucleotides and circular polynucleotides containing nucleotide sequence errors. Optionally, the target desired polynucleotide may be mutagenized across several distinct discrete regions by combining fragments each of which has been mutagenized across one discrete region as described.

“Sequence specific linear duplex polynucleotides” means a collection (two or more) of linear duplex polynucleotides or linear duplex oligonucleotides comprising two subsets of linear duplexes, wherein one strand of each duplex in the first subset can be chosen and placed end to end to form a circular permutation of the sense strand of the desired circular polynucleotide, and one strand of each duplex in the second subset can be chosen and placed end to end to form a circular permutation of the antisense strand of the desired circular polynucleotide, and wherein each end of each such chosen strand of each linear duplex in each such subset is interior to some such chosen strand of opposite sense from a linear duplex in the other subset, when aligned as reverse complements.

“Desired polynucleotide” means any DNA or RNA polynucleotide, or collection (i.e., two or more) of polynucleotides, that comprise(s) a desired sequence of nucleotides, including, without limitation, a polynucleotide that encodes a full length or truncated polypeptide of interest, a fusion or chimeric polypeptide of interest, one or more full or partial regulatory sequences such as a promoter, TATA box, origin of replication, ribosome binding site(s), transcription factor(s), silencer(s), enhancer(s), introns or transcription terminator(s), or a selectable marker(s) such as an antibiotic resistance gene. The desired polynucleotide may optionally comprise two or more homologous or heterologous polypeptides (such as, for example heavy and light antibody chains, or certain receptors, growth factors, or hormones comprising two or more polypeptides, such as insulin). The desired polynucleotide may optionally be a plasmid or other vector, such as a viral vector containing a gene of interest in addition to various transcription regulatory elements, selectable markers, and the like. The desired polynucleotide may optionally be genomic DNA, cDNA, or synthetic (coding and/or non-coding) DNA. The desired polynucleotide can be any length, as desired by one skilled in the art for the particular application. In some embodiments, particular advantages are provided by the methods herein when the desired polynucleotide is at least 100, 120, 140, 160, 180, 200, 220, 240, 260, 280, 300, 320, 340, 360, 380, 400, 450, 500, 600, 700, 800, 900, 1000, 1100, 1200, 1500, 1700, 2000, 2500 or 3000 base pair in length or more. As will be appreciated by those skilled in the art, the length of the desired polynucleotide is not limited in length to that which is available through solid phase or other chemical synthetic methods; the desired polynucleotide provided herein can be generated using, e.g., PCR or other enzyme-based polynucleotide synthetic methods, and, thus, can be much larger than that available for chemically synthesized oligonucleotides.

“Sequence Error” means a discrepancy between the actual and desired identities of the nucleotides encountered when a polynucleotide is traversed from the 5′ to the 3′ end.

“Mechanical Disruption” means to break one of the covalent links of the backbone of a polynucleotide by physical interaction, for example, by sonification.

The typical process for carrying out the present invention involves providing the sequence specific linear duplex polynucleotides that form a starting material for the present invention, heating the polynucleotides to melt the duplexes, and cooling the polynucleotides to allow reannealing where some of the polynucleotides will reanneal so as to form circular duplex polynucleotides. The circular duplex polynucleotide can be covalently bound in its circle form using a ligase. In some embodiments, the circular duplex polynucleotide can be covalently bound in its circle form using a polymerase, followed by a ligase. Any remaining linear single stranded or duplex polynucleotides can then be removed using exonuclease digestion, gel filtration chromatography, gel electrophoresis or other suitable standard separation methods. Circular duplexes containing nucleotide base pair mismatches can be destroyed via treatment with a specific mismatch-cleaving endonuclease, coupled with an exonuclease such that the resulting circular duplexes comprise the desired polynucleotide containing few, if any, nucleotide sequence errors. These circular complexes can then be used as templates for amplification as described below.

The skilled artisan will have previously identified the specific nucleotide sequence of each overlap in the sequence specific linear duplex polynucleotide or linear duplex oligonucleotide to be used to practice the invention. Typically, the sequence specific linear duplex molecules are DNA, however, where suitable, they may be RNA. The sequence specific linear duplex polynucleotide starting material may be comprised of chemically synthesized DNA (prepared via phosphoramidite synthesis, for example), cDNA clone(s), or genomic DNA. Thus, cDNA clones with previously identified specific end-primer sequences but not previously identified internal sequences can be used in accordance with the methods provided herein.

Depending on the size of each sequence specific linear duplex polynucleotide, it may be desirable or necessary to generate such sequence specific linear duplex polynucleotide from two or more oligonucleotides that are ligated together or used as templates for PCR amplification. For example, the sequence specific linear duplex polynucleotides can be prepared from self assembling oligonucleotides and then self assembled using methods such as those taught in U.S. Pat. No. 7,262,031 and U.S. Patent Publication Nos. 2005/0106590 and 2007/0009928.

Where more than one sequence specific linear duplex polynucleotide is to be used, approximately equimolar amounts of each such polynucleotide can be placed in a standard buffer (such as 50 mM Tris-HCL, pH 8, 100 mM NaCl and 1 mM EDTA) or a ligase buffer (10 mM MgCL2, 10 mM dithiothreitol, 1 mM ATP) to generate the starting reaction mixture.

The process for forming the circles is then initiated by heating the reaction mixture to a temperature that is sufficiently high to disrupt the duplexes. Typically, the temperature for heating will be in the range of 90-100 degrees Celsius, with 95-98 degrees Celsius commonly used. The skilled artisan can select the specific temperature to be used based on both the G-C content and length of each duplex polynucleotide. The reaction mixture can be heated for about 5-30 minutes, and is typically heated between 5 and 10 minutes. After heating, the reaction mixture can be cooled at a rate of about 1-5 degrees Celsius per minute, more typically at a rate of about one degree Celsius per minute. If the circular duplexes that form during the cooling period are to be ligated using a ligase, than the final cooling temperature is typically about 16-20 degrees Celsius. If the circular duplexes will not be treated with ligase, than the final cooling temperature can optionally be about 4 degrees Celsius.

In those instances where the circular duplex is to be ligated, such as where endo- and exo-nucleases are to be subsequently employed, a ligase such as bacterial T4 ligase, can be added and used according to the manufacturer's recommendations. As the skilled artisan will understand, other ligases used in the practice of biotechnology may be suitable for use herein, provided that they are used consistent with the manufacturer's instructions. Typically incubation in the presence of ligase will be at 10-25 degrees Celsius, preferably at about 16 degrees Celsius, and the period for incubation will be from about 30 minutes to 24 hours or more, depending on the ligase used.

After the annealing and ligation steps above, any remaining linear polynucleotides in the mixture (whether in duplex or single stranded form), can be removed by adding one or more exonucleases to the mixture. Examples of suitable exonucleases include, without limitation, lambda exonuclease, T7 exonuclease, exonuclease I, exonuclease III, and RecJ exonuclease. The exonucleases are typically used according to the manufacturer's instructions.

Endonuclease enzymes can be used destroy those circular duplex polynucleotides containing mismatches. Endonuclease treatment typically follows exonuclease treatment in such as way that exonuclease activity remains during endonuclease treatment; however, the two treatments may be conducted simultaneously. Examples of suitable endonucleases for use herein include, without limitation, endonuclease V, Bal 31 endonuclease, and mung bean nuclease. The endonucleases are typically used at a concentration and under conditions recommended by the manufacturer.

In those cases where it is desirable to amplify the desired polynucleotide, the circular duplex containing the desired polynucleotide can be directly amplified with the circular duplex as the template, or it can first be linearized using a restriction enzyme, mechanical disruption, or other known methods. PCR can be accomplished using oligonucleotide primers designed to amplify the desired polynucleotide. The resulting PCR reaction mixture can then be cloned into a suitable vector and the vector can be transformed into host cell. After culturing the host cells on agarose plates, colonies can be picked, grown in liquid culture medium, and their plasmids purified. The desired polynucleotide insert in the vector can then be sequence-verified using standard nucleic acid sequencing methods.

The methods of the present invention have several uses in the biotechnology field. For example, the methods permit the creation of duplex polynucleotides containing few, if any, errors. In addition, the methods allow for rapid amplification of polynucleotide molecules. Further, the methods allow the rapid creation of plasmids that can be inserted directly into host cells.

The following provides one example of the application of the invention disclosed herein, and, as will be understood by those skilled in the art, does not represent the limit or the full scope of the invention, but instead illustrates one application thereof.

### EXAMPLES

### Example 1

### Circular Assembly and Amplification of a 594 Base Pair Duplex Polynucleotide

The initial full-length 594 base pair gene (SEQ ID NO:31) was designed as a set of three primary DNA fragments overlapping each other by approximately 63 nucleotides each. Each primary DNA fragment was designed as a set of ten oligonucleotides with adjacent oligonucleotides overlapping each other by 20 to 30 nucleotides (Primary DNA fragment 1:243 base pairs, 10 oligonucleotides; Primary DNA fragment 2:240 base pairs, 10 oligonucleotides; Primary DNA fragment 3:237 base pairs, 10 oligonucleotides; Table 1 sets forth the oligonucleotides used; SEQ ID NOS:1-30). The oligonucleotides were purchased from Integrated DNA technologies, Inc. (Coralville, Iowa).

For each primary DNA fragment, the constituent oligonucleotide set was added to a primer extension reaction at a final concentration of about 0.02 mM along with an excess (about 0.2 μM) of leader and trailer primer oligonucleotides. (Primer Set for: Primary DNA fragment 1: On1 and On10; Primary DNA fragment 2: On9 and On18; Primary DNA fragment 3: On17 and On26).

Each oligonucleotide set was extended into a primary DNA fragment with about 2.5 U of PfuUltra High-Fidelity DNA polymerase (Stratagene, La Jolla, Calif.), 200 μM dNTPs (Roche Diagnostics, Indianapolis, Ind.), and 1× PfuUltra reaction buffer. These primer extension and PCR amplification reactions were performed using the following cycle: about 10 minutes of denaturation at about 95° C., followed by 30 cycles of about 20 seconds at about 95° C., about 20 seconds at about 50° C., and about 30 seconds at about 72° C., with a final step of about 5 minutes at about 72° C. Each of these constituent primary DNA fragments was then purified with a Qiagen PCR purification kit according to manufacturer's instructions (Qiagen, Valencia, Calif.).

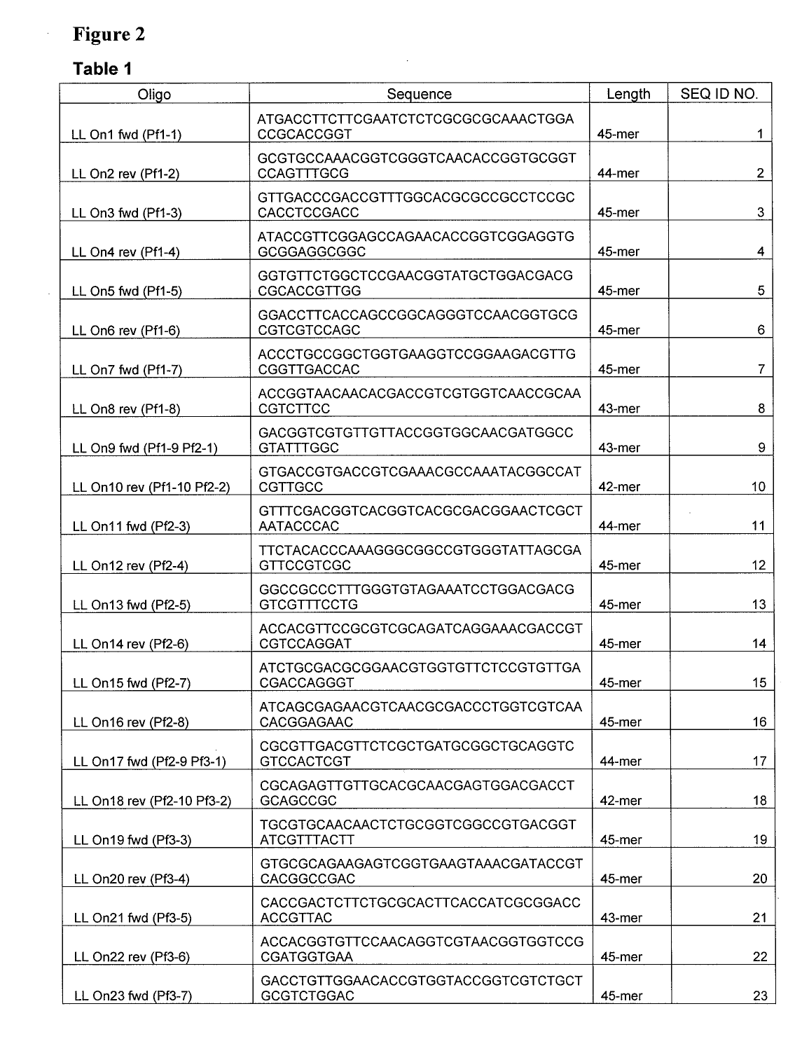

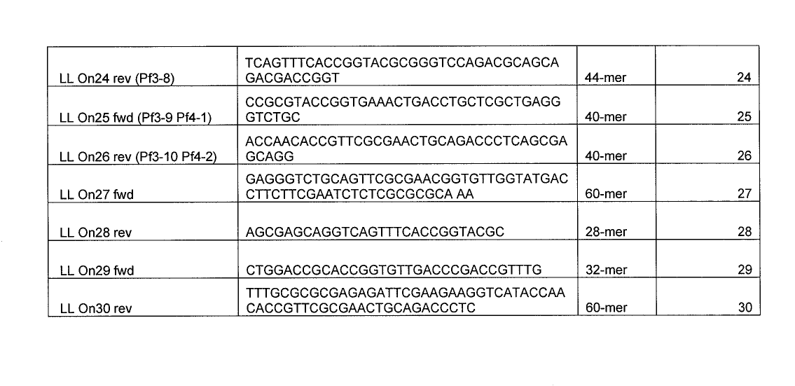

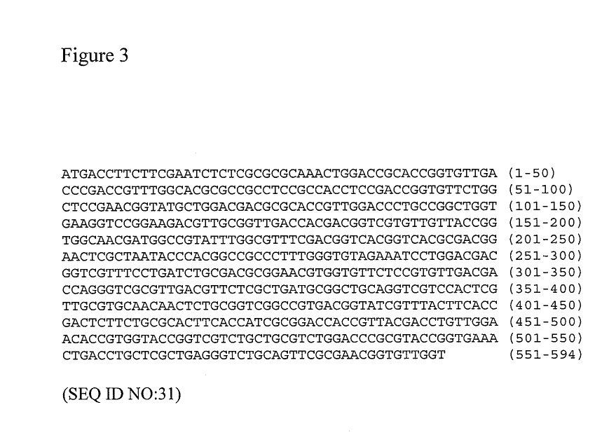

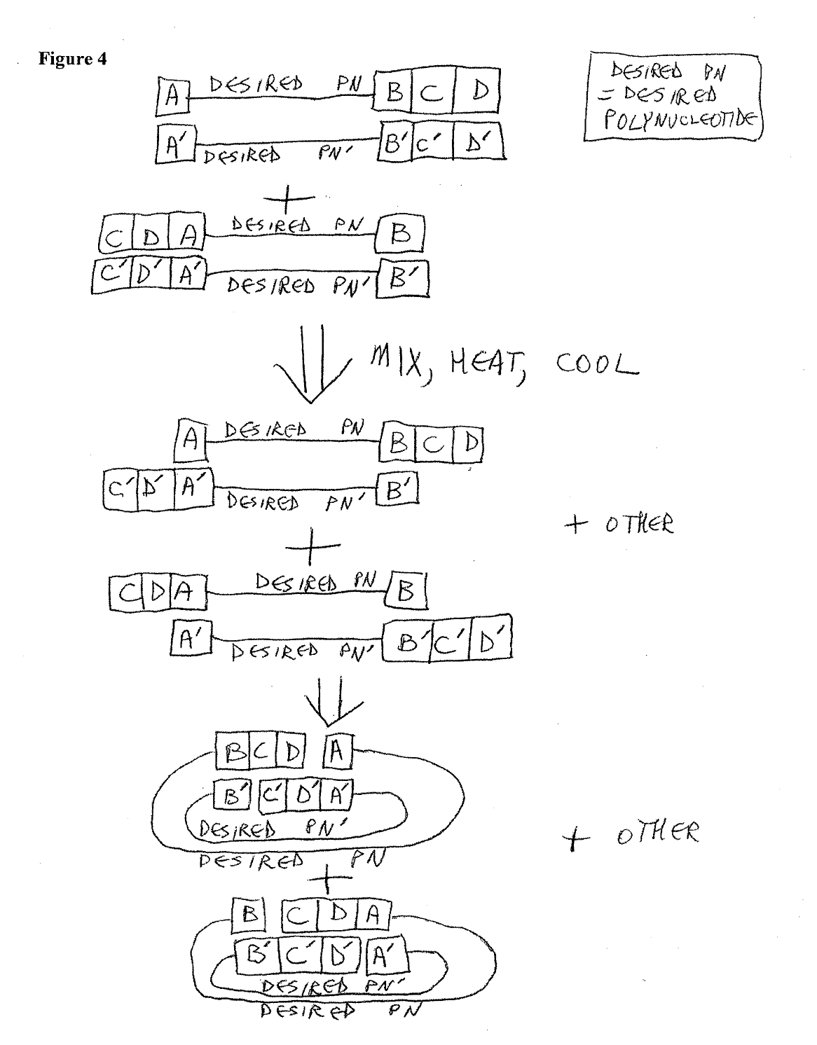

The constituent primary DNA fragment set was added to a primer extension reaction at a final concentration of about 0.02 μM along with an excess (0.2 μM) of leader and trailer primer oligonucleotides. (Primer Set for: Secondary DNA fragment 1: On1 and On26; FIG. 2 sets forth the sequences of the oligonucleotides used). The primary DNA fragment set was extended to the full-length 594 base pair gene using about 2.5 U of PfuUltra High-Fidelity DNA polymerase (Stratagene), 200 μM dNTPs (Roche Diagnostics, Indianapolis, Ind.), and 1× PfuUltra reaction buffer. These primer extension and PCR amplification reactions were performed using the following cycle: approximately 10 minutes of denaturation at 95° C., followed by 35 cycles of about 20 seconds at about 95° C., about 30 seconds at about 62° C., and about 1 minute at about 72° C., with a final step of about 5 minutes at about 72° C.

The resulting full-length gene was used as a template to generate sequence specific linear duplex polynucleotides for that gene, comprising two subsets here referred to as version 1 (“V1”) and version 2 (“V2”). To the primer extension and PCR reaction for the V1 linear duplex polynucleotide, 30 ng of the full length 594 base pair gene was used as a template with about 0.2 μM 5′phosphorylated oligonucleotide On27 and 5′phosphorylated oligonucleotide On28 primers. The oligonucleotide On27 contained, in the 5′ to 3′ direction, the last 30 bases of the full length gene followed by bases 1-30 of the full length gene, and the oligonucleotide On28 contained, in the 5′ to 3′ direction, the reverse complement of bases 537-564 of the full length gene. For the V2 linear duplex polynucleotide, 30 ng of the full length gene was used as a template with 0.2 μM 5′phosphorylated oligonucleotide On29 and 5′phosphorylated oligonucleotide On30 primers. The oligonucleotide On29 contained, in the 5′ to 3′ direction, bases 31-62 of the full length gene, and the oligonucleotide On30 contained, in the 5′ to 3′ direction, the reverse complement of bases1-30 of the full length gene, followed by bases 565-594 of the full length gene.

The V1 and V2 polynucleotides were isolated by primer extension and PCR amplification with about 2.5 U of PfuUltra High-Fidelity DNA polymerase (Stratagene), 200 μM dNTPs, and 1×PfuUltra buffer. These primer extension and PCR amplification reactions were performed using the following cycle: approximately 10 minutes of denaturation at about 95° C., followed by 30 cycles of about 20 seconds at about 95° C., about 30 seconds at about 60° C., and about 2 minutes at about 72° C., with a final step of about 5 minutes at about 72° C.

The V1 and V2 polynucleotides were combined in about 18 μl total reaction mixture to a final concentration of 0.0013 μM each. This mixture was incubated at about 98° C. for 10 minutes and ramped down to 16° C. at about 1° C./min. After the mixture reached about 16° C., approximately 2 μl of T4 DNA ligase (400 units/1 μl; New England Biolabs, Beverly, Mass.) was added to the reaction. The ligation reaction was incubated at about 16° C. overnight. This series of thermo-reactions was carried out using a thermocycler (Bio-Rad Engine Dyad Peltier Thermal Cycler). A negative control, which was incubated with about 2 μl of water instead of 2 μl of T4 DNA ligase, was run in parallel.

Exonucleases were used to degrade any remaining linear polynucleotides in the mixture. Approximately 00.5 μl aliquot of the circular assembly ligation reaction mixture was mixed in approximately 20 μl of total reaction volume, and contained about 1.1 μl of exonuclease III (100 U/μl, New England Biolabs, Beverly, Mass.), about 20.3 μl of exonuclease I (20 U/μl, New England Biolabs, Beverly, Mass.), about 1.1 μl of lambda exonuclease (100 U/μl, New England Biolabs, Beverly, Mass.), about 2 μl of 10× exonuclease buffer number 1 (New England Biolabs, Beverly, Mass.), and about 13 μl of water. The reaction was incubated at about 37° C. for about 4 hours.

After exonuclease incubation, each reaction mixture was split into four batches. The first batch (about 6 μl) was mixed with about 2 μl of water to a final volume of about 8 μl, and was incubated at about 37° C. overnight without any enzyme treatment. The second batch (about 4 μl) was mixed with about 0.5 μl of buffer number 4 (New England Biolabs, Beverly, Mass.) and about 1 μl of endonuclease V (10 U/μl, New England Biolabs, Beverly, Mass.). The third batch (about 4 μl) was mixed with about 1.5 μl of a cocktail made of about 10 μl of water, about 6 μl of buffer number 4 (New England Biolabs, Beverly, Mass.), and about 2.4 μl of endonuclease V (10 U/μl, New England Biolabs, Beverly, Mass.). The fourth batch was mixed with an approximately 1.5 μl of aliquot of a cocktail made of about 10 μl of water, about 6 μl of buffer 4 (New England Biolabs, Beverly, Mass.) and about 1.2 μl of endonuclease V (10 U/μl, New England Biolabs, Beverly, Mass.). All reactions were incubated overnight at about 37° C.

Following endonuclease treatment, each reaction mixture was heated at about 80° C. for about 20 minutes to heat-inactive all enzymes. Subsequently, about 0.3 μl of the restriction enzyme Kpn1 (10 U/ml, New England Biolabs, Beverly, Mass.) was added to each reaction mixture and incubated for about 1 hour at 37° C. to linearize the circular duplex polynucleotides. Each reaction mixture was then used as a template for the PCR amplification of the 594 base pair gene. PCR was accomplished using oligonucleotide primers On3 and On22 (see Table 1).

An aliquot of about 5.5 μl from each endonuclease reaction was used for the PCR amplification of the double stranded DNA. The PCR reaction was initiated by heating the mixture at about 95° C. for 10 minutes, followed by 30 cycles of the following program: about 95° C. for about 20 seconds, about 60° C. for about 30 seconds, and about 72° C. for about 60 seconds. A final extension at about 72° C. was carried out for about 10 minutes. Approximately 50 ng of this PCR reaction mixture was then used for cloning into the pCR-Blunt II-TOPO vector. Cloning was accomplished according to the manufacturer's instructions (Zero Blunt TOPO PCR Cloning Kit, Invitrogen, Carlsbad, Calif.). Individual colonies were picked and grown in Luria-Bertani broth containing about 50 ug/ml kanamycin. Plasmids from selected colonies were purified using the Qiagen QiA prep-spin mini-prep column (Qiagen, Valencia, Calif.), and the integrity of the 594 base pair gene sequence was verified by DNA sequencing (Cogenics Inc., Morrisville, N.C.).

Of 9,991 bases sequenced, three deletions were detected, and no insertions or mutations were seen. In contrast, with conventional production of the same gene one would expect to detect approximately 20 to 50 sequence errors in 9,991 bases sequenced, depending on the underlying chemical synthesis error rate.

All references cited herein are expressly incorporated by reference where permitted.

1. A method of making a circular duplex polynucleotide that comprises a desired polynucleotide, the method comprising:
(a) providing a mixture of sequence specific linear duplex polynucleotides; and
(b) denaturing and reannealing the polynucleotide mixture under conditions such that some of the polynucleotides form circular duplexes that comprise the desired polynucleotide.

2. A method of reducing nucleotide sequence errors in a pool of desired polynucleotides, comprising:
(a) providing a mixture of sequence specific linear duplex polynucleotides;
(b) denaturing and reannealing the polynucleotide mixture under conditions such that some of the polynucleotides form circular duplexes that comprise the desired polynucleotide;
(c) ligating circular duplexes to form covalently linked circular duplex polynucleotides; and
(d) removing linear polynucleotides and circular polynucleotides containing nucleotide sequence errors.

3. A method of cloning a desired polynucleotide, comprising:
(a), (b) performing methods (a) and (b) according to claim 1; and
(c) transforming the polynucleotide mixture of (b) into a host cell.

4. A method of reducing nucleotide sequence errors in a mutational library based on a desired polynucleotide, comprising:
(a) providing a mixture of linear duplex polynucleotides containing two circular permutations of the desired polynucleotide minus said mutagenesis region, one with and one without flanking regions surrounding the mutagenesis region;
(b) providing a mixture of single-stranded mutagenesis polynucleotides, each containing a variable mutagenesis region surrounded by said flanking regions;
(c) mixing, denaturing, and reannealing the mixtures under conditions such that some of the polynucleotides form circular duplex polynucleotides except that the mutagenesis region is single-stranded;
(d) extending the complementary strand across the single-stranded mutagenesis region by using a non-displacing polymerase;
(e) ligating circular duplexes to form covalently linked circular duplex polynucleotides; and
(f) destroying linear polynucleotides and circular polynucleotides containing nucleotide sequence errors.

5. The method of claim 2 further comprising amplifying the desired polynucleotide.

6. The method of claim 5 wherein amplification is accomplished by linearizing the circular duplexes and using polymerase chain reaction or polymerase extension for amplification.

7. The method of claim 1 wherein the denaturing and reannealing conditions comprise heating and then cooling the polynucleotide mixture.

8. The method of claim 7 wherein heating is conducted at a temperature from about 80-120 degrees Celsius for about 5-15 minutes, followed by cooling to about 10-20 degrees at a rate of about 0.5-2 degrees Celsius per minute.

9. The method of claim 8 wherein heating is at about 98 degrees Celsius for about 2 minutes, followed by cooling to about 16 degrees Celsius at a rate of about one degree per minute.

10. The method of claim 6 wherein the linear polynucleotides are removed using an exonuclease.

11. The method of claim 10 wherein the exonuclease is selected from the group consisting of: lambda exonuclease, T7 exonuclease, exonuclease I, exonuclease III, and RecJ exonuclease.

12. The method of claim 11 wherein the exonuclease is exonuclease III.

13. The method of claim 2 wherein circular duplex polynucleotides having nucleotide mismatches are removed using an endonuclease.

14. The method of claim 13 wherein the endonuclease is selected from the group consisting of: endonuclease V, Bal 31 endonuclease, and mung bean nuclease.

15. The method of claim 14 wherein the endonuclease is endonuclease V.

16. The method of claim 2 wherein linear polynucleotides and circular polynucleotides containing nucleotide sequence errors are removed using an exonuclease in the presence of an endonuclease.

17. The method of claim 16 wherein the exonuclease is selected from the group consisting of: lambda exonuclease, T7 exonuclease, exonuclease I, exonuclease III, and RecJ exonuclease; and the endonuclease is selected from the group consisting of: endonuclease V, Bal 31 endonuclease, and mung bean nuclease.

18. The method of claim 17 wherein the exonuclease is exonuclease III and the endonuclease is endonuclease V.

19. The method of claim 6 wherein linearization is accomplished using a ligase.

20. The method of claim 19 wherein the ligase is T4 DNA ligase.

21-31. (canceled)
